(19) United States
(12) Patent Application Publication
(10) Pub. No.: US 2015/0305921 A1
(43) Pub. Date: Oct. 29, 2015

(54) DEVICE AND METHOD FOR TREATING PROLAPSE

(71) Applicant: Lilian Audrey Taylor, Burnaby (CA)

(72) Inventor: Lilian Audrey Taylor, Burnaby (CA)

(21) Appl. No.: 14/643,203

(22) Filed: Mar. 10, 2015

Related U.S. Application Data
(60) Provisional application No. 61/950,678, filed on Mar. 10, 2014.

(51) Int. Cl. A61F 6/08; A61F 2/00

(52) U.S. Cl. CPC A61F 6/08; A61F 2/0009; A61F 2230/0008

(57) ABSTRACT

A device for treating pelvic floor prolapse comprises a) a substantially elliptical base comprising a concave surface and a convex surface; b) a contiguous apron extending outwardly and downwardly from the base, in all directions, and forming with the base, a cap-like structure, and wherein apron comprises a first short end, a second long end and two substantially symmetrical sides, all defining, along with the convex inner surface of the base, an interior hollow of the cap-like structure.

### FIELD OF THE INVENTION

The invention relates generally to the non-surgical treatment of urogenital conditions and more particularly, the invention relates to devices and non-surgical methods for use in treating female pelvic organ prolapse.

### BACKGROUND OF THE INVENTION

When intra-abdominal pressure pushes the vagina outside the body, vaginal prolapse can develop. In normal circumstances, various muscles close the pelvic floor, supporting it from below while fascia a ligaments support. Increases in abdominal pressure, failure of the muscles to keep the pelvic floor supported, and damage to the ligaments and fascia all contribute to the development of prolapse. In addition, if a woman has a hysterectomy, the vaginal angle may be altered and ligament support reduced, causing increased pressure at a more acute angle, accelerating the prolapse.

A variety of factors can cause genital prolapse in women. It is thought that individual women have differing inherent strength of the relevant connective tissue. The loss of connective tissue strength may be associated with damage at childbirth, deterioration with age, poor collagen repair mechanisms, and poor nutrition. Loss of muscle strength might also be associated with neuromuscular damage during childbirth, neural damage from chronic straining, and metabolic diseases that affect muscle function. Other factors involved in prolapse include increased loads on the supportive system, as seen in prolonged lifting or chronic coughing from chronic pulmonary disease, or some disturbance in the balance of the structural support of the genital organs. Possible factors may also include obesity, constipation, and a history of hysterectomy.

While not generally life-threatening, vaginal prolapse and the concomitant anterior cystocele can lead to discomfort, urinary incontinence, and incomplete emptying of the bladder. Posterior vaginal prolapse may additionally cause defecatory problems, such as tenesmus and constipation. A range of symptoms are associated with pelvic organ prolapse such as a feeling of pressure from the organs pressing against the vaginal wall, feeling very full in the lower abdomen, urinary incontinence, or inability to empty the bladder completely, pain in the vagina, constipation, incomplete or difficult bowel emptying, pain, and the like.

Current treatment options range from dietary and lifestyle changes and exercises to strengthen the pelvic muscles (Kegels) for mild symptoms; use of a pessary for more severe cases; to surgery for serious cases.

The surgical options include anterior vaginal wall plasty, vaginal total hysterectomy, posterior vaginal wall plasty, Manchester operation, sacrospinous ligament fixation of vaginal apex, musculus iliococcygeus fasciodesis (Inmon procedure) and sacrouterine ligament fixation (McCall technique). This latter procedure involves attaching the vaginal vault to the sacrospinous ligament, which requires specialized skills and has the disadvantage of tending to place the vagina in an artificial anatomical position.

These are mostly (highly) invasive procedures requiring general or spinal anaesthesia and prolonged operative time. While surgery can be effective the patient is subjected to considerable pain during the surgery and healing as well as being at risk of complications such as incontinence, urinary retention, pain during sex, infection, bladder or bowel injury, erosion or the formation of a fistula. Furthermore, the prolapse can return after surgery and, on average, 30% of subjects having surgery will require a further surgery within 4 years. In addition, in a three year period in the United States and Canada the use of the mesh has been associated with over 1,000 adverse events prompting the FDA and Health Canada to issue public health notifications regarding the “serious complications” associated with transvaginal implantation of mesh for the treatment of pelvic organ prolapse.

Furthermore, these procedures also require significant surgical expertise in the area of pelvic reconstructive surgery. The surgery and follow-on care during the patient's recovery both tend to be relatively expensive and are particularly burdensome for women due to pain, vaginal discharge or bleeding, and the prolonged time required for bladder and bowel function to return. These operations all require long training periods and extensive learning curves to become competent. As a result, they are usually performed by a small minority of gynecologists, not available in every region.

Sling procedures are commonly used to treat prolapse conditions. A sling procedure is a surgical method involving the placement of a sling to stabilize or support the bladder neck or urethra. There are a variety of different sling procedures. Slings used for pubovaginal procedures differ in the type of material and anchoring methods. In some cases, the sling is placed under the bladder neck and secured via suspension sutures to a point of attachment (e.g. bone) through an abdominal and/or vaginal incision. Complications of sling procedures include urethral obstruction, development of de novo urge incontinence, hemorrhage, prolonged urinary retention, infection, and damage to surrounding tissue and sling erosion.

Insofar as non-surgical options are concerned, the pessary is often used. In one example, it is a toroidal shaped ring which is inserted into vagina and prevents the organs dropping by lifting up vagina. However the pessaries are not without risks--there can be complications due to vaginal wall ulceration, among other issues.

It is clear that 1) prolapse is a huge and growing problem (due to aging female baby boomers) and 2) there is a need for a minimally invasive yet highly effective device and method that can be used to treat pelvic organ prolapse with no or virtually no side effects. Such a device should reduce the complexity of procedures that are currently available while being biocompatible, adjustable, and non-toxic.

There is much room for improvement in this area. It is an object of the present invention to obviate or mitigate the above noted and other disadvantages.

### SUMMARY OF THE INVENTION

The present invention provides a device for treating pelvic floor prolapse which comprises a) a substantially elliptical base comprising a concave surface and a convex surface; b) a contiguous apron extending outwardly and downwardly from the base, in all directions, and forming with the base, a cap-like structure, and wherein apron comprises a first short end, a second long end and two substantially symmetrical sides, all defining, along with the convex inner surface of the base, an interior hollow of the cap-like structure; and wherein said device is in situ in a patient: i) the concave outer surface of the base abuts and supports the pelvic floor; and ii) an extended rim of the apron, distal to the base, holds the device in place and supports to the base by engagement with female labia, exterior to the vagina.

The present invention further provides a method of treating pelvic floor prolapse in a user which comprises the user securing device in labia, said device comprising a) a substantially elliptical base comprising a concave surface and a convex surface; b) a contiguous apron extending outwardly and downwardly from the base, in all directions, and forming with the base, a cap-like structure, and wherein apron comprises a first short end, a second long end and two substantially symmetrical sides, all defining, along with the convex inner surface of the base, an interior hollow of the cap-like structure; and wherein i) the concave outer surface of the base abuts and supports the pelvic floor; and ii) an extended rim of the apron, distal to the base, holds the device in place and supports to the base by engagement with female labia, exterior to the vagina.

The present invention further provides a method of treating pelvic floor prolapse in a user which comprises the user securing the device in her labia, wherein said device comprises a) a substantially elliptical base comprising a concave surface and a convex surface; b) a contiguous apron extending outwardly and downwardly from the base, in all directions, and forming with the base, a cap-like structure, and wherein apron comprises a first short end, a second long end and two substantially symmetrical sides, all defining, along with the convex inner surface of the base, an interior hollow of the cap-like structure; and wherein, upon placement by user: i) the concave outer surface of the base abuts and supports the user's pelvic floor; and ii) an extended rim of the apron, distal to the base, holds the device in place and supports to the base by engagement with the user's labia, exterior to her vagina.

The device of the invention offers an alternative to internal assistive devices such as pessaries, with their attendant disadvantages. It is secured and worn external to the vagina and as such, problems such as vaginal wall ulceration, improperly fitted devices are all avoided.

There is provided herein an alternative treatment option for prolapse which remedies the condition in a safe and durable manner and which is inexpensive, simple to use, minimally invasive, carries a reduced chance of infection, reduced incidence of pain and reduced complications in use.

### BRIEF DESCRIPTION OF THE DRAWINGS

FIG. 1a is a perspective view of prior art pessary device in situ;

FIG. 1b is a perspective view of various prior art pessary devices;

FIG. 1c is a perspective view of two prior art pessary devices;

FIG. 2 is a perspective view (from top) of the prolapse treating device of the invention;

FIG. 3 is a top plan view of the prolapse treating device of the invention;

FIG. 4 is a side view of the prolapse treating device of the invention;

FIG. 5 is a perspective view (from top and side) of the prolapse treating device of the invention;

FIG. 6 is a perspective view (from side and bottom) of the prolapse treating device of the invention;

FIG. 7 is a perspective view (from front and side) of a further embodiment of a prolapse treating device of the invention, wherein material is quilted and absorbent;

FIG. 8 is a perspective view (from bottom and side) of a further embodiment of a prolapse treating device of the invention, wherein material is quilted and absorbent.

The figures depict an embodiment of the present invention for purposes of illustration only. One skilled in the art will readily recognize from the following description that alternative embodiments of the structures and methods illustrated herein may be employed without departing from the principles of the invention described herein.

### DETAILED DESCRIPTION OF THE PREFERRED EMBODIMENTS

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured

### I. Terms

The term “method” means any process, method or the like, unless expressly specified otherwise.

Each process (whether called a method or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

The term “invention” and the like mean “the one or more inventions disclosed in this application”, unless expressly specified otherwise.

The terms “an aspect”, “an embodiment”, “embodiment”, “embodiments”, “the embodiment”, “the embodiments”, “one or more embodiments”, “some embodiments”, “certain embodiments”, “one embodiment”, “another embodiment” and the like mean “one or more (but not all) embodiments of the disclosed invention(s)”, unless expressly specified otherwise.

The term “variation” of an invention means an embodiment of the invention, unless expressly specified otherwise.

A reference to “another embodiment” or “another aspect” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase “at least one of a widget, a car and a wheel” means either (i) a widget, (ii) a car, (iii) a wheel, (iv) a widget and a car, (v) a widget and a wheel, (vi) a car and a wheel, or (vii) a widget, a car and a wheel. The phrase “at least one of”, when such phrase modifies a plurality of things does not mean “one of each of” the plurality of things.

Numerical terms such as “one”, “two”, etc. when used as cardinal numbers to indicate quantity of something (e.g., one widget, two widgets), mean the quantity indicated by that numerical term, but do not mean at least the quantity indicated by that numerical term. For example, the phrase “one widget” does not mean “at least one widget”, and therefore the phrase “one widget” does not cover, e.g., two widgets.

The phrase “based on” does not mean “based only on”, unless expressly specified otherwise. In other words, the phrase “based on” describes both “based only on” and “based at least on”. The phrase “based at least on” is equivalent to the phrase “based at least in part on”.

The term “represent” and like terms are not exclusive, unless expressly specified otherwise. For example, the term “represents” do not mean “represents only”, unless expressly specified otherwise. In other words, the phrase “the data represents a credit card number” describes both “the data represents only a credit card number” and “the data represents a credit card number and the data also represents something else”.

The term “whereby” is used herein only to precede a clause or other set of words that express only the intended result, objective or consequence of something that is previously and explicitly recited. Thus, when the term “whereby” is used in a claim, the clause or other words that the term “whereby” modifies do not establish specific further limitations of the claim or otherwise restricts the meaning or scope of the claim.

The term “pessary” means a medical device inserted into the vagina, either to provide structural support, or as a method of delivering medication.

The term “e.g.” and like terms mean “for example”, and thus does not limit the term or phrase it explains. For example, in a sentence “the computer sends data (e.g., instructions, a data structure) over the Internet”, the term “e.g.” explains that “instructions” are an example of “data” that the computer may send over the Internet, and also explains that “a data structure” is an example of “data” that the computer may send over the Internet. However, both “instructions” and “a data structure” are merely examples of “data”, and other things besides “instructions” and “a data structure” can be “data”.

The term “respective” and like terms mean “taken individually”. Thus if two or more things have “respective” characteristics, then each such thing has its own characteristic, and these characteristics can be different from each other but need not be. For example, the phrase “each of two machines has a respective function” means that the first such machine has a function and the second such machine has a function as well. The function of the first machine may or may not be the same as the function of the second machine.

The term “i.e.” and like terms mean “that is”, and thus limits the term or phrase it explains. For example, in the sentence “the computer sends data (i.e., instructions) over the Internet”, the term “i.e.” explains that “instructions” are the “data” that the computer sends over the Internet.

As used herein, the term “treatment” means any manner in which a pelvic organ prolapse in a subject is ameliorated, or otherwise beneficially altered. This includes bladder prolapse.

As used herein, the terms “subject”, “user” and “patient” may be used interchangeably.

As used herein, “a” or “an” means “at least one” or “one or more”.

### II. Further Detail

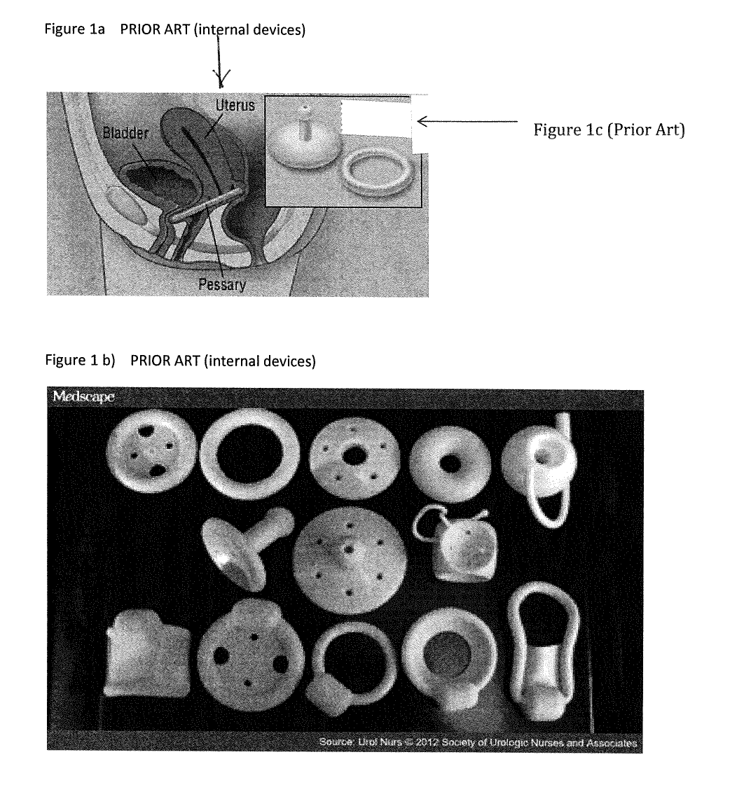

Turning to the drawings, wherein like numbers denote like parts throughout the several views, FIGS. 1a, 1b and 1c are illustrative views of prior art pessaries. More specifically, reference is made to Figure la in which it is made clear that these pessaries are used and placed entirely completely within the vagina. FIG. 1b illustrates the shapes and types of these interior use devices.

Many “internal use” pessary styles and sizes are available, and FIGS. 1a, 1b and 1c display a variety of current pessaries. While successfully fitted pessaries offer some symptom reduction and quality-of-life improvement for some users, no specific shape of pessary is best for all women. Pessary size selection is also currently guided by experience because no specific vaginal measures have predicted successful fit. In one study, Pelvic Organ Prolapse Quantification (POP-Q), parameters were tested as a potential objective predictor but were not found to predict pessary size; however, women with a shorter total vaginal length were less likely to be successfully fittedl. Thus, pessary fitting remains an art, with some reliance on trial and error. Because there has been little success in identifying objective evidence to improve pessary choice, new providers must rely on expert opinion and mentorship. ¹ Nager, C. W., Richter, H. E., Nygaard, I., Paraiso, M. F., Wu, J. M., Kenton, K., . . . Spino, C., for the Pelvic Floor Disorders Network. (2009). POP-Q measures do not predict incontinence pessary size. International Urogynecology Journal, 20, 1023-1028. doi:10.1007/s00192-009-0866-1.

Pessary manufacturers provide recommendations to help the novice match pessary styles with patient findings². Successful fitting also depends on clinician experience and training. Few nursing or medical programs spend time teaching pessary use³. If a Ring pessary is not successful, but there is some introital support, a Donut or Inflatable-Donut pessary may be an option. Donut pessaries are essentially thicker Rings and may fill a vagina enlarged by loss of elasticity more completely; however, these pessaries are in more complete contact with the vaginal epithelium, which may increase the risk of mechanical tissue injury or discharge production and retention. Some women may be able to remove and replace traditional Donut pessaries themselves, but comfortable vaginal intercourse is unlikely. Inflatable Donut pessaries, which are a silicone variation of an older latex inflatable pessary called the Inflatoball, have a stem and valve for inflation and deflation like a balloon, and are designed to be inserted and removed at frequent intervals (within 48 hours).

The overall picture, in regards to the internal use pessaries of FIG. 1 is that there remain significant inconveniences and drawbacks to their use.

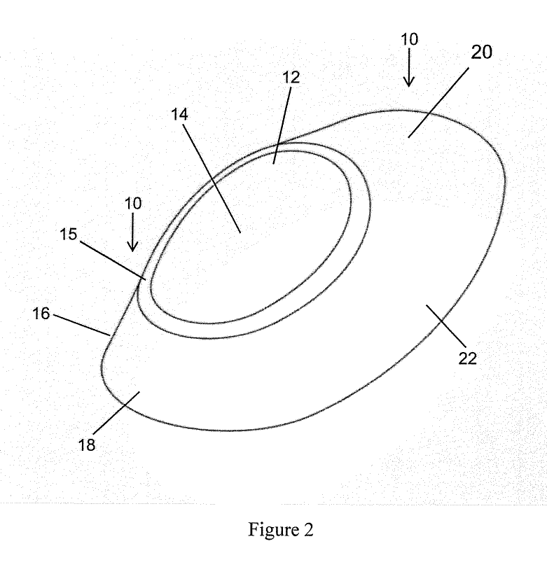

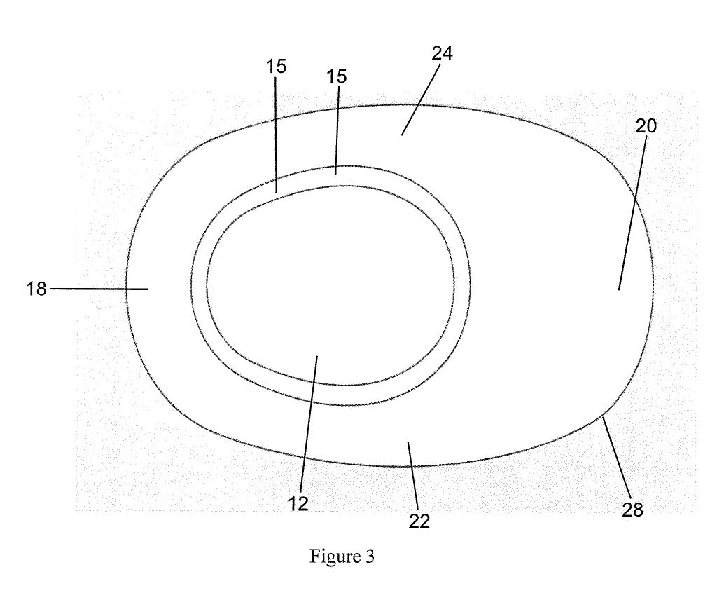

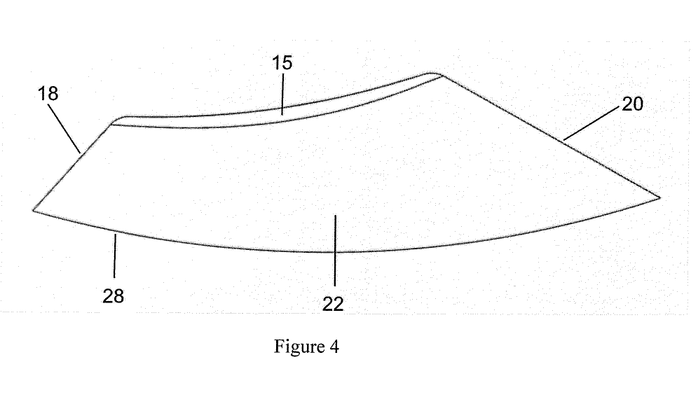

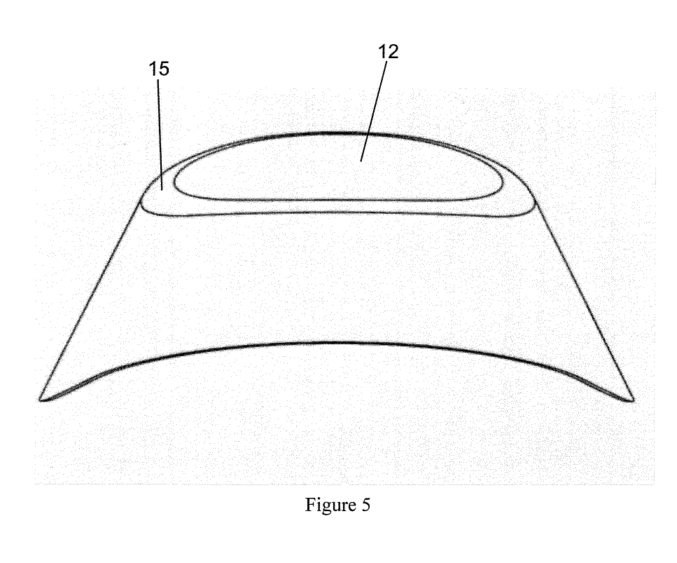

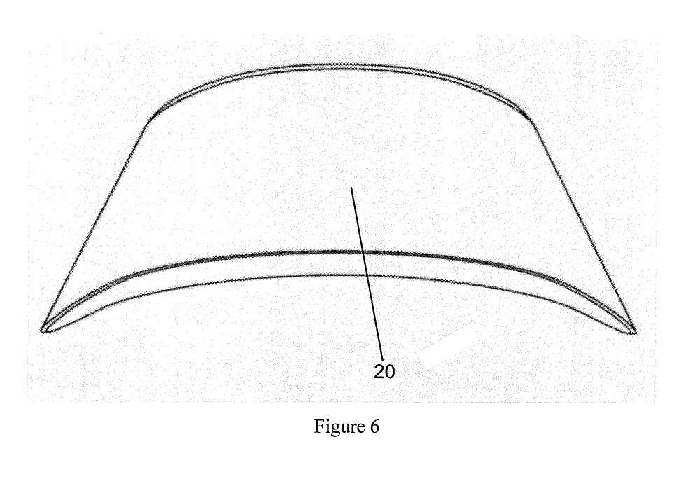

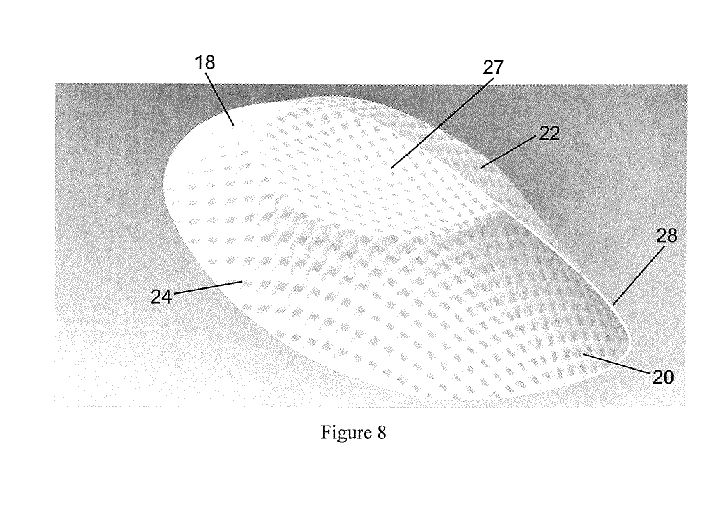

FIGS. 2 to 8 represent preferred devices in accordance with the present invention. FIG. 2 shows a device generally at 10 which comprises a substantially elliptical base 12 comprising a concave surface 14 and a contiguous apron 16 extending outwardly and downwardly from the base, in all directions, and forming with the base, a cap-like structure. Rim 15 defines a boundary between base 12 and apron 16. Apron 16 is shown to comprise a first short end 18, a second long end 20 and two substantially symmetrical sides (22 and 24), all defining, along with a convex inner surface 27 (shown best in FIG. 8) of the base, an interior hollow 26 of the cap-like structure. ²Bioteque of America. (2011). Pessary products. Retrieved from http://www.bioteque.com/downloads/index3Pott-Grinstein, E., & Newcomer, J. R. (2001). Gynecologists' patterns of prescribing pessaries. Journal of Reproductive Medicine, 46(3), 205-208.

Extended rim 28 of apron 16, distal to base 12, holds the device in place and supports to the base by engagement with female labia, exterior to the vagina. It can be seen, in particular in FIGS. 2, 3, 4 and 8, apron 16 is shown to comprise the first short end 18, the second long end 20 and two substantially symmetrical sides (22 and 24). In this way apron 16 defines the elliptical shape required for secure placement and holding of the device, by a female user's own labia. The device is inserted with second long end 20 directed to or “pointing” towards front of female user's vagina and first short end 18 directed to or “pointing” towards the rear. Extended rim 28 of apron 16, in fact, engages (when device is in situ) within folds of labia, comfortably holds the device in place, and therein facilitates the abutment of concave surface 14 with pelvic floor.

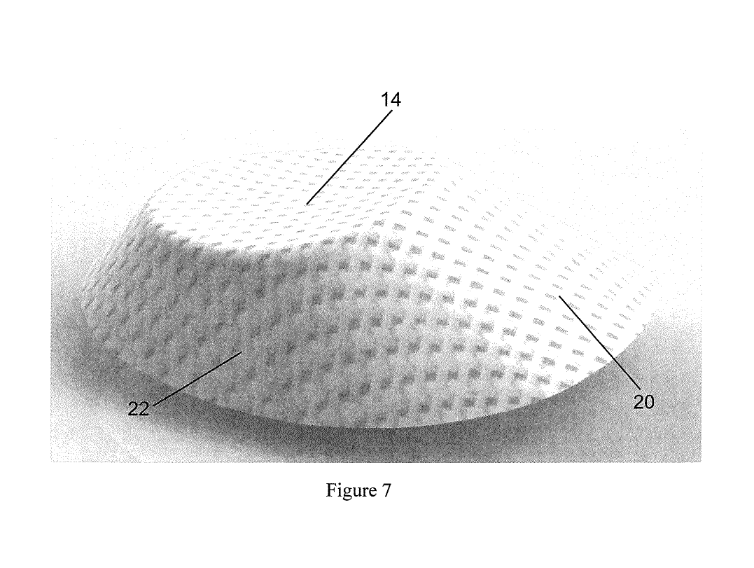

Device is made of a biologically inert material. Preferably plastic or silicone are used. A cotton or other absorbent type material may be employed, as shown (in a weaved quilted fashion) in FIGS. 7 and 8. The device may be made of non-woven fabrics, such as rayon. It is to be understood that many materials can be used and formed into the the shape of the device of the invention.

“Non-woven fabric” shall mean a material having a fibrous identity whose individual fibers have been entangled by fluid forces applied to the fibers through a fluid-permeable structure which conditions and regulates the fluid forces, whereby the fluid forces mechanically interlock the fibers to form a unitary structure having a predetermined pattern) constituting a primary liquid-retaining component, like material used to form a panty liner. However, it is to be understood that the device of the invention, while flexible enough to bend and insert, retains sufficient integrity: 1) at the base to uphold the pelvic floor and 2) at the apron and rim, to hold the device in place in the labia. Surprisingly, not a great deal of rigidity is required to accomplish this.

The device of the invention may be of varying sizes. Due to simplicity of manufacture, it is anticipated that a user will be able to use the particular device best suited to her body anatomically. That said, it is likely that three main sizes will suffice for a majority of the population.

In one preferred aspect, the device is approximately 3 to 4 inches in length (longest axis of ellipse, which is greatest distance across extended rim 28) , more preferably 3-½″ in length. In one preferred aspect, the device is approximately 0.5 to 2 inches in width (across interior hollow 26, which is across width of extended rim 28), more preferably 1″ in width.

In one preferred aspect, the device is approximately 1 to 2 inches in depth, more preferably with a 1-¼″ deep side wall.

It is preferred that the base is from 1-2 inches long and from 0.5 to 1.5 inches wide, more preferably 1-½″ long and ¾″ wide.

III. Operation and Use

In operation, the following steps are taken by a user in need of some degree of pelvic floor support. The easiest analogy is to the placement of an “elliptical” cap within the labia—wherein user places thumb or finger against convex inner surface 27 and gently pushes device towards but not through opening of vagina. Extended rim 28 of apron 16 engages within folds of labia, (user can adjust for comfort) and comfortably holds the device in place, and therein facilitates the abutment of concave surface 14 with pelvic floor. User will immediately feel a secure connection between concave surface 14 and her pelvic floor. This is the first and only product which provides this security without the full insertion of a pessary within the vagina.

As will be apparent to those skilled in the art, the various embodiments described above can be combined to provide further embodiments. Aspects of the present systems, methods and components can be modified, if necessary, to employ systems, methods, components and concepts to provide yet further embodiments of the invention. For example, the various devices and methods described above may omit some parts or acts, include other parts or acts, and/or execute acts in a different order than set out in the illustrated embodiments.

Further, in the methods taught herein, the various acts may be performed in a different order than that illustrated and described. Additionally, the methods can omit some acts, and/or employ additional acts.

I claim:

1. A device for treating pelvic floor prolapse which comprises:
a) a substantially elliptical base comprising a concave surface and a convex surface;
b) a contiguous apron extending outwardly and downwardly from the base, in all directions, and forming with the base, a cap-like structure, and wherein apron comprises a first short end, a second long end and two substantially symmetrical sides, all defining, along with the convex inner surface of the base, an interior hollow of the cap-like structure;
and wherein said device is in situ in a patient: i) the concave outer surface of the base abuts and supports the pelvic floor; and ii) an extended rim of the apron, distal to the base, holds the device in place and supports to the base by engagement with female labia, exterior to the vagina.

2. The device of claim 1 wherein base and apron are unitary.

3. The device of claim 1 which is biologically inert.

4. The device of claim 1 which is made of one of silicone or plastic.

5. The device of claim 1 which is made of cotton or other absorbent type material.

6. The device of claim 1 which is made of a non-woven fabric.

7. The device of claim 1 which is approximately 3 to 4 inches in length (longest axis of ellipse, which is greatest distance across extended rim) , and approximately 0.5 to 2 inches in width (across the interior hollow, which is across width of extended rim).

8. The device of claim 1 which is approximately 1 to 2 inches in depth.

9. The use of a device of claim 1 in treating pelvic floor prolapse.

10. A method of treating pelvic floor prolapsed by placement of the device of claim 1 in a female user's labia.

11. A method of treating pelvic floor prolapse in a user which comprises the user securing device in her labia, wherein said device comprises a) a substantially elliptical base comprising a concave surface and a convex surface; b) a contiguous apron extending outwardly and downwardly from the base, in all directions, and forming with the base, a cap-like structure, and wherein apron comprises a first short end, a second long end and two substantially symmetrical sides, all defining, along with the convex inner surface of the base, an interior hollow of the cap-like structure; and wherein, upon placement by user: i) the concave outer surface of the base abuts and supports the user's pelvic floor; and ii) an extended rim of the apron, distal to the base, holds the device in place and supports to the base by engagement with the user's labia, exterior to her vagina.

12. The method of claim 11 wherein device base and apron are unitary.

13. The method of claim 11 wherein device is biologically inert.

14. The method of claim 11 wherein device is made of one of silicone or plastic.

15. The method of claim 11 wherein device is made of cotton or other absorbent type material.

16. The method of claim 11 wherein device is made of a non-woven fabric.

17. The method of claim 11 wherein device is approximately 3 to 4 inches in length (longest axis of ellipse, which is greatest distance across extended rim) , and approximately 0.5 to 2 inches in width (across the interior hollow, which is across width of extended rim).

18. The method of claim 11 wherein device is approximately 1 to 2 inches in depth.
